(19) United States
(12) Patent Application Publication
(10) Pub. No.: US 2021/0090718 A1
(43) Pub. Date: Mar. 25, 2021

(54) LABELING APPARATUS AND METHOD, AND MACHINE LEARNING SYSTEM USING THE LABELING APPARATUS

(71) Applicant: Vingroup Joint Stock Company, Ha Noi City (VN)

(72) Inventor: Steven Quoc Hung TRUONG, La Jolla, CA (US)

(21) Appl. No.: 16/923,775

(22) Filed: Jul. 8, 2020

(30) Foreign Application Priority Data
Sep. 25, 2019 (VN) 1-2019-05241

(51) Int. Cl. G16H 30/40

(52) U.S. Cl. CPC G16H 30/40

(57) ABSTRACT

A labeling apparatus according to an embodiment of the present invention includes a pathological data receiving unit configured to receive pathological data about a patient when a first label is a positive index or an uncertain index in a medical image of the patient which is labeled with the first label, a label input unit configured to receive a second label corresponding to the pathological data, a medical image receiving unit configured to receive a medical image corresponding to the pathological data, and a first processing unit configured to label the received medical image with the positive index and store the medical image labeled with the positive index in a positive data set when the second label is the positive index, and label the received medical image with a negative index and store the medical image labeled with the negative index in a negative data set when the second label is the negative index.

### CROSS-REFERENCE TO RELATED APPLICATIONS

This application claims priority to Vietnamese Patent Application No. 1-2019-05241 filed on Sep. 25, 2019, which is incorporated herein by reference in its entirety.

### BACKGROUND

### 1. Field of the Invention

Embodiments of the present invention relate to a labeling apparatus and method for a medical image, and a machine learning system using the labeling apparatus.

### 2. Discussion of Related Art

According to recent research by the Radiological Society of North America (RSNA), the error rate for radiation diagnostic is around 30%. For example, the diagnostic error rate for lung cancer with a median nodule diameter of 16 mm is 19% and the diagnostic error rate for breast cancer is 30%. Due to the diagnostic errors, medical images of about 20 million people are misdiagnosed each year and 10% of the misdiagnosed people die. However, the number of radiologists who analyze medical images is extremely limited especially in under developed countries. Statistics show that around 4.7 billion people worldwide are not able to access radiologists.

In order to solve this problem, an algorithm for providing diagnostic medical images as a doctor assistant, i.e. a secondary care doctor are being developed. In particular, there is a growing interest in the research and development of machine learning-based algorithms.

The machine learning-based algorithm is an algorithm in which accuracy of determination is improved as learning is performed using training data. However, the accuracy of the determination is not necessarily improved as the learnt algorithm is performed. In order to improve the accuracy of the determination, accuracy of the training data that trains the algorithm must be high degree of accuracy. When the algorithm is trained using inaccurate training data, the accuracy of the determination is lowered for supervised learning algorithm.

In order to generate the training data, a method in which a label is extracted from a radiology report and an X-ray image is labeled with the extracted label using a natural language processing (NLP) tool is used. The above method may be sufficient for generating a large amount of training data but dataset is not sufficient due to doctors' and the NLP tool errors. However, since errors in natural language processing tools or errors in radiology reports are applied without a method to clean them up, the quality of the training data is low.

The human brain can be divided into ‘learning’ that accumulates various knowledge and information in the head, and ‘reasoning’ that derives answers to new information based on that knowledge. Artificial intelligence, such as machine learning and deep learning, also performs “training” and “reasoning” like human learning processes.

Training is an essential process for reasoning. Without training there is no reasoning. If there is an error in training, reasoning becomes inaccurate. On the other hand, if there is no error in training, reasoning is correct. Therefore, the most important component of AI is training.

It is the training data that determines the performance of training in AI. Just as humans make accurate reasoning when learning from a large amount of high-quality information, artificial intelligence can improve the accuracy of reasoning by learning with large amounts of high-quality training data.

In the case of artificial intelligence in medicine, the accuracy of reasoning is very important. Because wrong reasoning can take a person's life. In particular, in the case of medical image diagnosis, a large amount of high-quality training data is further required because the probability of treatment of a disease varies greatly according to the accuracy of reasoning.

### SUMMARY OF THE INVENTION

The present invention provides a labeling apparatus and method thereof for achieving the above object. Embodiments of the present invention is the novel data mining and knowledge mining method on how to obtain high accuracy (nearly 100%) as well as achievable faster 100× during the labeling process. Thus, a high quality training data for medical images can be provided, and a machine learning system for diagnostic medical image algorithm is improved based on learning from the generated training data.

Objects to be solved in the embodiments are not limited thereto, and objects or effects that can be grasped from the aspects or embodiments described below will also be included.

According to an aspect of the present invention, there is provided a labeling apparatus including a pathological data receiving unit configured to receive pathological data about a patient when a first label is a positive index or an uncertain index in a medical image of the patient which is labeled with the first label, a label input unit configured to receive a second label corresponding to the pathological data, a medical image receiving unit configured to receive a medical image corresponding to the pathological data, and a first processing unit configured to label the received medical image with the positive index and store the medical image labeled with the positive index in a positive data set when the second label is the positive index, and label the received medical image with a negative index and store the medical image labeled with the negative index in a negative data set when the second label is the negative index.

The medical image may include an X-ray image and the pathological data may include computed tomography (CT) scan information and biopsy information.

The medical image receiving unit may receive the medical images in the order in which a time point at which the medical image is captured is closest to a time point at which the pathological data is generated.

The labeling apparatus may further include a health information input unit configured to receive health information about the patient when the first label is the negative index in the medical image of the patient which is labeled with the first label, and a second processing unit configured to store the medical image labeled with the first label in the negative data set when the health information includes a normal index and store the medical image labeled with the first label in an uncertain data set when the health information includes an abnormal index.

The health information input unit may receive the health information about the patient when the second label is the negative index and a confidence level of the pathological data is lower than a reference level, and the second processing unit may store the medical image labeled with the second label in the negative data set when the received health information includes the normal index and store the medical image labeled with the second label in the uncertain data set when the received health information includes the abnormal index.

According to another aspect of the present invention, there is provided a labeling method including receiving pathological data about a patient when a first label is a positive index or an uncertain index in a medical image of the patient which is labeled with the first label, receiving a second label corresponding to the pathological data, receiving a medical image corresponding to the pathological data, labeling the medical image with the positive index and storing the medical image labeled with the positive index in a positive data set when the second label is the positive index, and labeling the medical image with a negative index and storing the medical image labeled with the negative index in a negative data set when the second label is the negative index.

The medical image may include an X-ray image and the pathological data may include CT scan information and biopsy information.

The receiving of the medical image may include receiving the medical images in the order in which a time point at which the medical image is captured is closest to a time point at which the pathological data is generated.

The method may further include receiving health information about the patient when the first label is the negative index in the medical image of the patient which is labeled with the first label, storing the medical image labeled with the first label in the negative data set when the health information includes a normal index, and storing the medical image labeled with the first label in an uncertain data set when the health information includes an abnormal index.

The method may further include receiving the health information about the patient when the second label is the negative index and a confidence level of the pathological data is less than a reference level, storing the medical image labeled with the second label in the negative data set when the received health information includes the normal index, and storing the medical image labeled with the second label in an uncertain data set when the received health information includes the abnormal index.

According to still another aspect of the present invention, there is provided a machine learning system including a labeling apparatus configured to generate a first medical image and a first training data set which is generated based on pathological data corresponding to the first medical image, a medical diagnosis apparatus configured to compare first medical opinion information about a user with respect to a second medical image to second medical opinion information about a machine learning-based medical image diagnosis algorithm and generate a second training data set by labeling the matched medical opinion information on the medical image when the first medical opinion information and the second medical opinion information match and by labeling third medical opinion information which is input by the user on the medical image when the first medical opinion information and the second medical opinion information do not match, and a machine learning apparatus configured to cause the machine learning-based medical image diagnosis algorithm to learn on the basis of the first training data set and the second training data set.

The labeling apparatus may include a pathological data receiving unit configured to receive pathological data about a patient when a first label is a positive index or an uncertain index in the first medical image of the patient which is labeled with the first label, a label input unit configured to receive a second label corresponding to the pathological data, a first medical image receiving unit configured to receive a first medical image corresponding to the pathological data, and a first processing unit configured to label the received first medical image with the positive index and store the first medical image labeled with the positive index in a positive data set when the second label is the positive index, and label the received first medical image with a negative index and store the first medical image labeled with the negative index in a negative data set when the second label is the negative index.

According to yet another aspect of the present invention, there is provided a recording medium configured to store a program for executing any one of the above methods.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a configuration diagram of a machine learning system according to an embodiment of the present invention;

FIG. 2 is a flowchart for describing a machine learning method using a machine learning system according to an embodiment of the present invention;

FIGS. 3 and 4 are configuration diagrams of labeling apparatuses according to embodiments of the present invention;

FIG. 5 is a flowchart for describing a labeling method according to an embodiment of the present invention;

FIG. 6 is a flowchart illustrating in detail a process (S500) of FIG. 5 according to an embodiment of the present invention;

FIG. 7 is a flowchart illustrating in detail the process (S500) of FIG. 5 according to another embodiment of the present invention; and

FIG. 8 is a flowchart illustrating in detail a process (S600) of FIG. 5 according to an embodiment of the present invention.

### DETAILED DESCRIPTION OF EXEMPLARY EMBODIMENTS

While the present invention may have various modifications and alternative forms, specific embodiments thereof are shown by way of example in the drawings and will be described herein in detail. However, there is no intent to limit the present invention to the particular forms disclosed. On the contrary, the present invention is to cover all modifications, equivalents, and alternatives falling within the spirit and scope of the appended claims.

It should be understood that, although the terms “first,” “second,” and the like may be used herein to describe various elements, the elements are not limited by the terms. The terms are only used to distinguish one element from another element. For example, a first element could be termed a second element, and, similarly, a second element could be termed a first element without departing from the scope of the present invention. As used herein, the term “and/or” includes any and all combinations of one or more of the associated listed items.

It should be understood that when an element is referred to as being “connected” or “coupled” to another element, the element may be directly connected or coupled to another element or intervening elements may be present. In contrast, when an element is referred to as being “directly connected” or “directly coupled” to another element, there are no intervening elements present.

The terminology used herein is for the purpose of describing particular embodiments only and is not intended to be limiting to the present invention. As used herein, the singular forms “a,” “an,” and “the” are intended to also include the plural forms, unless the context clearly indicates otherwise. It should be further understood that the terms “comprise,” “comprising,” “include,” and/or “including,” when used herein, specify the presence of stated features, integers, steps, operations, elements, parts, or combinations thereof, but do not preclude the presence or addition of one or more other features, integers, steps, operations, elements, parts, or combinations thereof.

Unless otherwise defined, all terms including technical and scientific terms used herein have the same meaning as commonly understood by one of ordinary skill in the art to which this invention belongs. It should be further understood that terms, such as those defined in commonly used dictionaries, should be interpreted as having a meaning that is consistent with their meaning in the context of the relevant art and are not to be interpreted in an idealized or overly formal sense unless expressly so defined herein.

Hereinafter, embodiments will be described in detail with reference to the accompanying drawings, the same or corresponding components are denoted by the same reference numerals regardless of reference numbers, and thus the description thereof will not be repeated.

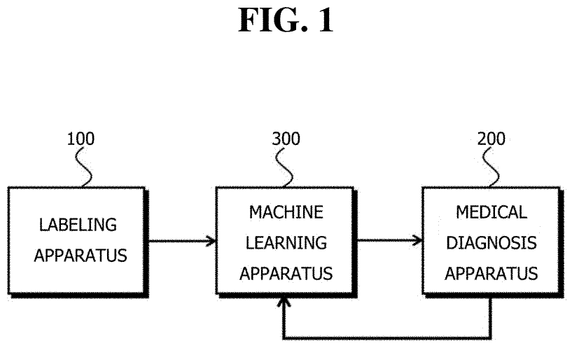

FIG. 1 is a configuration diagram of a machine learning system according to an embodiment of the present invention.

The machine learning system according to the embodiment of the present invention relates to a learning system of a medical image diagnosis algorithm that can aid a doctor in making medical determination by providing a medical opinion on the basis of a medical image.

Referring to FIG. 1 the machine learning system according to the embodiment of the present invention may include a labeling apparatus 100, a medical diagnosis apparatus 200, and a machine learning apparatus 300.

First, the labeling apparatus 100 may be an apparatus for labeling a medical image with a label and generating a training data set.

The labeling apparatus 100 may generate a first medical image and a first training data set which is generated based on pathological data corresponding to the first medical image. The first medical image may be an X-ray image and the pathological data may be computed tomography (CT) scan information and biopsy information. The labeling apparatus 100 will be described below in detail with reference to the accompanying drawings.

Next, the medical diagnosis apparatus 200 may provide medical opinion information on the basis of a second medical image. Here, the second medical image may be an image which is identical to the first medical image. The second medical image may be an X-ray image. Specifically, the medical diagnosis apparatus 200 may compare first medical opinion information of a user with respect to the second medical image to second medical opinion information of a machine learning-based medical image diagnosis algorithm. When the first medical opinion information and the second medical opinion information match, the medical diagnosis apparatus 200 may label the medical image with the matched medical opinion information. On the other hand, when the first medical opinion information and the second medical opinion information do not match, the medical diagnosis apparatus 200 may label the second medical image with third medical opinion information which is input by the user. The medical image, which is labeled with the matched medical opinion information or the third medical opinion information, may be generated and stored as a second training data set.

The machine learning-based medical image diagnosis algorithm may include a supervised learning-based machine learning algorithm. The machine learning-based medical image diagnosis algorithm may include a gradient descent algorithm, logistic regression, k-nearest neighbor (KNN), a support vector machine (SVM), a doctor decision tree, convolutional neural networks (CNNs), recurrent neural networks (RNNs), or the like.

Next, the machine learning apparatus 300 may cause the machine learning-based medical image diagnosis algorithm to learn on the basis of the training data set. The machine learning apparatus 300 may cause the machine learning-based medical image diagnosis algorithm to learn on the basis of the first training data set and the second training data set.

The labeling apparatus 100 and the medical diagnosis apparatus 200 may be implemented through personal computers (PCs), tablet PCs, servers, or the like. A user who uses the labeling apparatus 100 and the medical diagnosis apparatus 200 may be a radiologist who professionally analyzes medical images. The labeling apparatus 100 and the medical diagnosis apparatus 200 may be implemented through the same terminal. For example, the labeling apparatus 100 and the medical diagnosis apparatus 200 may be implemented through the same PC.

The machine learning apparatus 300 may be implemented through a PC, a tablet PC, a server, or the like. The machine learning apparatus 300 may be connected to the labeling apparatus 100 and/or the medical diagnosis apparatus 200 via wired and/or wireless communication networks. The machine learning apparatus 300 may receive the training data sets from the labeling apparatus 100 and/or the medical diagnosis apparatus 200 via the wired and/or wireless communication networks.

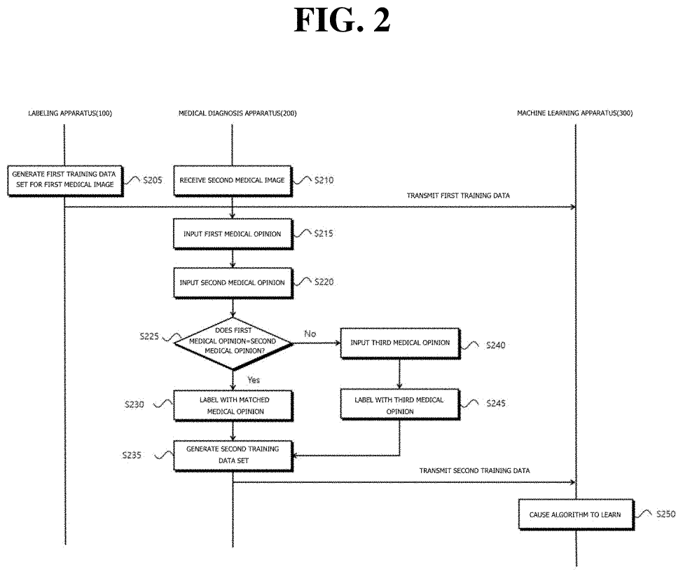

FIG. 2 is a flowchart for describing a machine learning method using a machine learning system according to an embodiment of the present invention.

Referring to FIG. 2, a labeling apparatus 100 generates a first training data set for a first medical image (S205). Further, the labeling apparatus 100 transmits the generated first training data set to a machine learning apparatus 300.

A medical diagnosis apparatus 200 receives a second medical image (S210). The first medical image and the second medical image may be different images or may be the same image.

The medical diagnosis apparatus 200 receives first medical opinion information and second medical opinion information in response to the second medical image (S215 and S220). The first medical opinion information may refer to a medical opinion for the second medical image, which is input by a user. For example, when a doctor examines the second medical image and as a result, determines that there is cardiomegaly in second medical image, the medical diagnosis apparatus 200 may receive cardiomegaly as the first medical opinion information. The second medical opinion information may refer to a medical opinion for the second medical image, which is determined by the machine learning-based medical image diagnosis algorithm.

The medical diagnosis apparatus 200 may compare the first medical opinion information to the second medical opinion information (S225). When the first medical opinion information and the second medical opinion information match, the medical diagnosis apparatus 200 labels the second medical image with the matched medical opinion (S230) and generates a second training data set (S235). For example, when both of the first medical opinion information and the second medical opinion information for the second medical image are determined as cardiomegaly, the medical diagnosis apparatus 200 labels the second medical image with cardiomegaly and generates the second training data set.

On the other hand, when the first medical opinion information and the second medical opinion information do not match, the medical diagnosis apparatus 200 may receive third medical opinion information (S240), label the second medical image with the third medical opinion information (S245), and generate the second training data set (S235). In this case, the third medical opinion information may be input by the user. The third medical opinion information may be identical to or different from the first medical opinion information or the second medical opinion information. For example, when the first medical opinion information indicates that there is cardiomegaly and the second medical opinion information indicates that there is no disease, the doctor may input pneumonia as the third medical opinion information about the second medical image.

The generated second training data set is transmitted to the machine learning apparatus 300.

The machine learning apparatus 300 causes the machine learning-based medical image diagnosis algorithm to learn on the basis of the first training data set and the second training data set (S250). The machine learning apparatus 300 may cause the machine learning-based medical image diagnosis algorithm to learn and then transmit the learned medical image diagnosis algorithm to the medical diagnosis apparatus 200. That is, the medical diagnosis apparatus 200 may update the medical image diagnosis algorithm on the basis of a learned result.

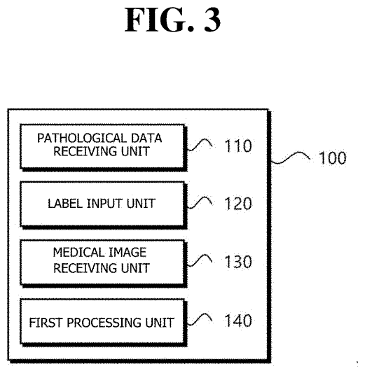

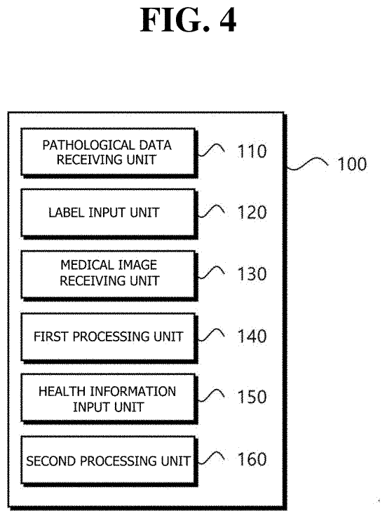

FIGS. 3 and 4 are configuration diagrams of labeling apparatuses according to embodiments of the present invention.

Referring to FIG. 3, a labeling apparatus 100 according to an embodiment of the present invention may include a pathological data receiving unit 110, a label input unit 120, a medical image receiving unit 130, and a first processing unit 140. Further, referring to FIG. 4, a labeling apparatus 100 according to another embodiment of the present invention may further include a health information input unit 150 and a second processing unit 160.

The pathological data receiving unit 110 may receive pathological data about a patient when a first label is a positive index or an uncertain index in a medical image of the patient which is labeled with the first label.

The medical image may include an X-ray image. The pathological data may include CT scan information and biopsy information.

The medical image labeled with the first label may be generated using a first label which is input by a person who professionally examines the medical image. For example, a radiologist may review a medical image and then enter a review result as a first label, and thus the first label may be labeled on the medical image.

The first label may be any one of a positive index, an uncertain index, and a negative index. The positive index may be an index indicating that there is a disease. The positive index may indicate that there is a disease such as cardiomegaly and pneumonia. The negative index may be an index indicating that there is no disease. The uncertain index may refer to an index indicating that the determination of existence of a disease is difficult. That is, the uncertain index may refer to an index indicating that the determination of existence of a disease is deferred.

The label input unit 120 may receive a second label corresponding to the pathological data. For example, it is assumed that as a biopsy result, colon cancer is found. Then, the label input unit 120 may receive the second label, “colon cancer.”

The medical image receiving unit 130 may receive a medical image corresponding to the pathological data. The medical image receiving unit 130 may receive the medical images in the order in which a time point at which the medical image is captured is closest to a time point at which the pathological data is generated.

The first processing unit 140 may label the medical image with a label according to the second label and store the labeled medical image. When the second label is a positive index, the first processing unit 140 may label the medical image which receives the positive index and store the medical image labeled with the positive index in a positive data set. On the other hand, when the second label is a negative index, the first processing unit 140 may label the medical image which receives the negative index and store the medical image labeled with the negative index in a negative data set.

The health information input unit 150 may receive health information about the patient when the first label is the negative index in the medical image of the patient which is labeled with the first label.

Further, the health information input unit 150 may receive the health information about the patient when the second label is the negative index and a confidence level of the pathological data is less than a reference level.

The second processing unit 160 may store the medical image labeled with the first label in the negative data set when the health information includes a normal index. On the other hand, the second processing unit 160 may store the medical image labeled with the first label in an uncertain data set when the health information includes an abnormal index.

Further, the second processing unit 160 may label the medical image which receives the negative index and store the medical image labeled with the negative index in the negative data set when the second label is the negative index and the confidence level of the pathological data is equal or greater than the reference level. On the other hand, the second processing unit 160 may store the medical image labeled with the second label in the negative data set when the received health information includes the normal index. On the other hand, the second processing unit 160 may store the medical image labeled with the second label in the uncertain data set when the health information includes the abnormal index.

The data stored in the positive data set and the data stored in the negative data set may be used to cause the machine learning-based medical image diagnosis algorithm to learn. However, the data stored in the uncertain data set may not be used to cause the medical image diagnosis algorithm to learn. The uncertain data set may be used later for radiologists to conduct in-depth analysis.

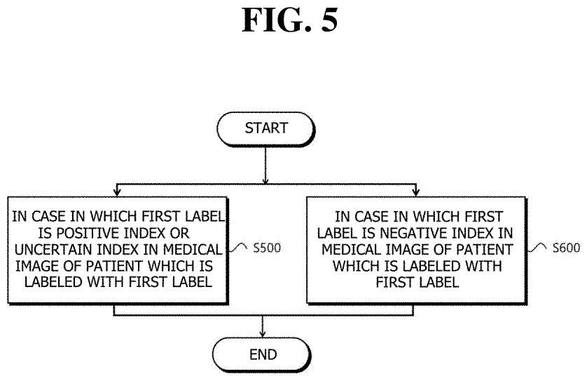

FIG. 5 is a flowchart for describing a labeling method according to an embodiment of the present invention.

The labeling method according to the embodiment of the present invention may be performed using the labeling apparatus 100 according to the embodiment of the present invention.

Referring to FIG. 5, the labeling method according to the embodiment of the present invention may be divided into a first process S500 and a second process S600. The first process S500 and the second process S600 may be selected according to a type of a first label labeled on a medical image.

The first process S500 is a process which is performed when the first label is a positive index or an uncertain index in a medical image of a patient which is labeled with the first label. That is, the first process S500 may be performed when disease information is labeled on the medical image or when content indicating that the presence of the disease is uncertain is labeled on the medical image.

The second process S600 is a process which is performed when the first label is a negative index in the medical image of the patient which is labeled with the first label. That is, the second process S600 may be performed when content indicating that there is no disease is labeled on the medical image.

Contents of the first process S500 and the second process S600 will be described below in detail with reference to the accompanying drawings.

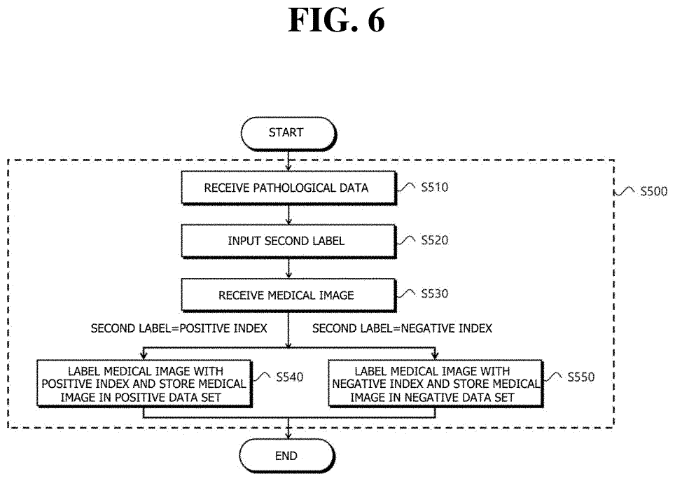

FIG. 6 is a flowchart illustrating in detail the first process S500 of FIG. 5 according to an embodiment of the present invention.

First, when the first label is the positive index or the uncertain index in the medical image of the patient which is labeled with the first label, the pathological data receiving unit 110 receives the pathological data about the patient (S510).

Further, the label input unit 120 receives the second label corresponding to the pathological data (S520).

Further, the medical image receiving unit 130 receives the medical image corresponding to the pathological data (S530).

Next, when the second label is the positive index, the first processing unit 140 labels the medical image which receives the positive index and stores the medical image labeled with the positive index in the positive data set (S540).

However, when the second label is the negative index, the first processing unit 140 labels the medical image which receives the negative index and stores the medical image labeled with the negative index in the negative data set (S550).

Meanwhile, a plurality of medical images may be captured between a time point at which the medical image is first captured and a time point at which the pathological data is generated. Therefore, the medical image may be provided as a plurality of medical images, and the processes S530 to S550 may be repeatedly performed on all the medical images. In this case, the medical images may be received and processed in the order in which a time point at which the medical image is captured is closest to a time point at which the pathological data is generated.

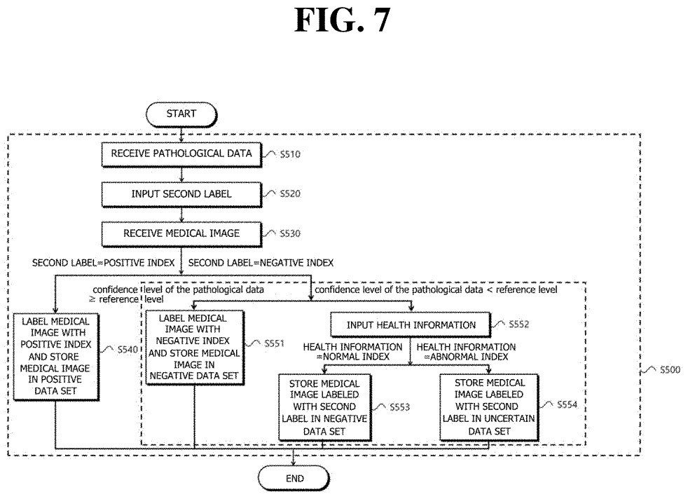

FIG. 7 is a flowchart illustrating in detail the first process S500 of FIG. 5 according to another embodiment of the present invention.

Since processes S510 to S540 illustrated in FIG. 7 are identical to the processes S510 to S540 described in FIG. 6, detailed descriptions thereof will be omitted. In the embodiment illustrated in FIG. 7, the process S550 illustrated in FIG. 6 is subdivided.

In the labeling apparatus 100 according to the embodiment of the present invention, the processes are divided according to the confidence level of the pathological data when the second label is the negative index. Here, the confidence level of the pathological data may be received together with the pathological data when the pathological data is received or may be separately received after the pathological data is received. The reference level may be preset and may be changed by the user.

First, when the confidence level of the pathological data is equal or greater than to the reference level, the health information input unit 150 may label the received medical image with the negative index and store the medical image labeled with the negative index in the negative data set (S551). The process S551 may be identical to the process S550 illustrated in FIG. 6.

On the other hand, when the confidence level of the pathological data is lower than the reference level, the health information input unit 150 receives the health information about the patient (S552).

In this case, when the received health information includes the normal index, the second processing unit 160 may store the medical image labeled with the second label in the negative data set (S553). On the other hand, when the received health information includes the abnormal index, the second processing unit 160 may store the medical image labeled with the second label in the uncertain data set (S554).

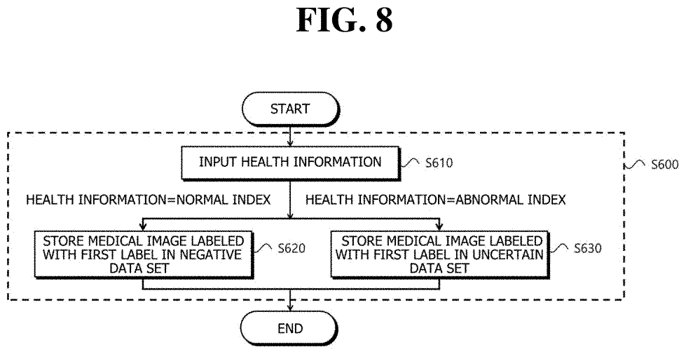

FIG. 8 is a flowchart illustrating in detail the second process S600 of FIG. 5 according to an embodiment of the present invention.

When the first label is the negative index in the medical image of the patient which is labeled with the first label, the health information input unit 150 receives the health information about the patient (S610).

In this case, when the health information includes the normal index, the second processing unit 160 may store the medical image labeled with the first label in the negative data set (S620). On the other hand, when the health information includes the abnormal index, the second processing unit 160 may store the medical image labeled with the first label in the uncertain data set (S630).

According to the embodiment of the present invention, the present invention may be implemented as a recording medium which stores a program for executing any one of the above-described methods.

High accuracy labeling of medical images may be performed.

According to the embodiment, a learning level of the machine learning-based medical image diagnosis algorithm may be significantly improved using highly accurate training data.

Terms described in the specification such as “unit” refer to software or a hardware component such as a field-programmable gate array (FPGA) or an Application-Specific Integrated Circuit (ASIC), and the unit performs certain functions. However, the “unit” is not limited to software or hardware. The “unit” may be configured in a storage medium that may be addressed or may be configured to be executed by at least one processor. Therefore, examples of the “unit” include components such as software components, object-oriented software components, class components and task components, and processes, functions, attributes, procedures, subroutines, segments of program codes, drivers, firmware, micro codes, circuits, data, database, data structures, tables, arrays, and variables. Components and functions provided from “units” may be combined into a smaller number of components and “units” or may be further separated into additional components and “units.” In addition, the components and the “units” may be implemented to playback one or more central processing units (CPUs) in a device or a secure multimedia card.

According to the embodiment, high accuracy labeling of medical images can be performed.

According to the embodiment, a learning level of the machine learning-based medical image diagnosis algorithm can be significantly improved using highly accurate training data.

Various and advantageous effects of the present invention are not limited to the above description and will be more easily understood in describing specific embodiments of the present invention.

While the present invention has been described with reference to the embodiments, the embodiments are only exemplary embodiments of the present invention and do not limit the present invention, and those skilled in the art will appreciate that various modifications and applications, which are not exemplified in the above description, may be made without departing from the scope of the essential characteristic of the present exemplary embodiments. For example, each component described in detail in the embodiments can be modified. In addition, it should be understood that differences related to these modifications and applications are within the scope of the present invention as defined in the appended claims.

What is claimed is:

1. A labeling apparatus comprising:
a pathological data receiving unit configured to receive pathological data about a patient when a first label is a positive index or an uncertain index in a medical image of the patient which is labeled with the first label;
a label input unit configured to receive a second label corresponding to the pathological data;
a medical image receiving unit configured to receive a medical image corresponding to the pathological data; and
a first processing unit configured to label the received medical image with the positive index and store the medical image labeled with the positive index in a positive data set when the second label is the positive index, and label the received medical image with a negative index and store the medical image labeled with the negative index in a negative data set when the second label is the negative index.

2. The labeling apparatus of claim 1, wherein:
the medical image includes an X-ray image; and
the pathological data includes computed tomography (CT) scan information and biopsy information.

3. The labeling apparatus of claim 1, wherein the medical image receiving unit receives the medical images in the order in which a time point at which the medical image is captured is closest to a time point at which the pathological data is generated.

4. The labeling apparatus of claim 1, further comprising:
a health information input unit configured to receive health information about the patient when the first label is the negative index in the medical image of the patient which is labeled with the first label; and
a second processing unit configured to store the medical image labeled with the first label in the negative data set when the health information includes a normal index and store the medical image labeled with the first label in an uncertain data set when the health information includes an abnormal index.

5. The labeling apparatus of claim 4, wherein:
the health information input unit receives the health information about the patient when the second label is the negative index and a confidence level of the pathological data is lower than a reference level; and
the second processing unit stores the medical image labeled with the second label in the negative data set when the received health information includes the normal index and stores the medical image labeled with the second label in the uncertain data set when the received health information includes the abnormal index.

6. A labeling method comprising:
receiving pathological data about a patient when a first label is a positive index or an uncertain index in a medical image of the patient which is labeled with the first label;
receiving a second label corresponding to the pathological data;
receiving a medical image corresponding to the pathological data;
labeling the medical image with the positive index and storing the received medical image labeled with the positive index in a positive data set when the second label is the positive index; and
labeling the medical image with a negative index and storing the received medical image labeled with the negative index in a negative data set when the second label is the negative index.

7. The labeling method of claim 6, wherein:
the medical image includes an X-ray image; and
the pathological data includes computed tomography (CT) scan information and biopsy information.

8. The labeling method of claim 6, wherein the receiving of the medical image includes receiving the medical images in the order in which a time point at which the medical image is captured is closest to a time point at which the pathological data is generated.

9. The labeling method of claim 6, further comprising:
receiving health information about the patient when the first label is the negative index in the medical image of the patient which is labeled with the first label;
storing the medical image labeled with the first label in the negative data set when the health information includes a normal index; and
storing the medical image labeled with the first label in an uncertain data set when the health information includes an abnormal index.

10. The labeling method of claim 9, further comprising:
receiving the health information about the patient when the second label is the negative index and a confidence level of the pathological data is less than a reference level;
storing the medical image labeled with the second label in the negative data set when the received health information includes the normal index; and
storing the medical image labeled with the second label in an uncertain data set when the received health information includes the abnormal index.

11. A machine learning system comprising:
a labeling apparatus configured to generate a first medical image and a first training data set which is generated based on pathological data corresponding to the first medical image;
a medical diagnosis apparatus configured to compare first medical opinion information about a user with respect to a second medical image to second medical opinion information about a machine learning-based medical image diagnosis algorithm and generate a second training data set by labeling the matched medical opinion information on the medical image when the first medical opinion information and the second medical opinion information match and by labeling third medical opinion information which is input by the user on the medical image when the first medical opinion information and the second medical opinion information do not match; and
a machine learning apparatus configured to cause the machine learning-based medical image diagnosis algorithm to learn on the basis of the first training data set and the second training data set.

12. The machine learning system of claim 11, wherein the labeling apparatus includes:
a pathological data receiving unit configured to receive pathological data about a patient when a first label is a positive index or an uncertain index in the first medical image of the patient which is labeled with the first label;
a label input unit configured to receive a second label corresponding to the pathological data;
a first medical image receiving unit configured to receive a first medical image corresponding to the pathological data; and
a first processing unit configured to label the received first medical image with the positive index and store the first medical image labeled with the positive index in a positive data set when the second label is the positive index, and label the received first medical image with a negative index and store the first medical image labeled with the negative index in a negative data set when the second label is the negative index.

13. A recording medium configured to store a program for executing the method of claim 6.
